(19) United States
(12) Patent Application Publication
(10) Pub. No.: US 2009/0287020 A1
(43) Pub. Date: Nov. 19, 2009

(54) PROCESS FOR PRODUCING HIGH-PURITY N-VINYLCARBOXAMIDES

(76) Inventors: Hiroshi Uchida, Oginachi (JP); Tetsuya Ishii, Kawasaki-shi (JP)

Correspondence Address: SUGHRUE MION, PLLC, 2100 PENNSYLVANIA AVENUE, N.W., SUITE 800, WASHINGTON, DC, 20037, US

(21) Appl. No.: 12/063,518

(22) PCT Filed: Aug. 4, 2006
(86) PCT No.: PCT/JP2006/315894

§ 371 Date: Feb. 11, 2008

Related U.S. Application Data
(60) Provisional application No. 60/719,198, filed on Sep. 22, 2005.

(30) Foreign Application Priority Data
Aug. 11, 2005 (JP) 2005-233452

(51) Int. Cl. C07C 233/01

(52) U.S. Cl. 564/206

(57) ABSTRACT

Disclosed is a process for producing high-purity N-vinylcarboxamides comprising: (A) a step of dissolving a crude N-vinylcarboxamide in an alcohol having 1 to 3 carbon atoms, the crude N-vinylcarboxamide containing 50 to 97% by mass of an N-vinylcarboxamide; (B) a step of adding an aliphatic hydrocarbon having 5 to 10 carbon atoms to the composition obtained in the step (A) to precipitate a crystal of the N-vinylcarboxamide; and (C) a step of separating the crystal of the N-vinylcarboxamide precipitated in the step (B).

### CROSS-REFERENCE TO RELATED APPLICATIONS

This application claims the benefit under 35 U.S.C. .sctn. 119(e) of U.S. Provisional Application No. 60/719,198 filed on Sep. 22, 2005.

### TECHNICAL FIELD

The present invention relates to a process for producing high-purity N-vinylcarboxamides. More specifically, the invention relates to an inexpensive process for producing high-purity N-vinylcarboxamides having high polymerizability.

### BACKGROUND ART

As known in the art, N-vinylcarboxamides are produced by pyrolysis or catalytic cracking of N-(1-alkoxyethyl) carboxamides synthesized from carboxamides, acetaldehydes and alcohols. Also known is a process in which ethylidenebiscarboxamides are synthesized from carboxamides and acetaldehydes, and the ethylidenebiscarboxamides are decomposed into carboxamides and N-vinylcarboxamides.

The former process affords N-vinylcarboxamides which contain unreacted materials such as carboxamides and N-(1-alkoxyethyl)carboxamides. The latter process yields equimolar amounts of N-vinylcarboxamide and carboxamide, and the product is a mixture of these. Because N-vinylcarboxamides, carboxamides and N-(1-alkoxyethyl) carboxamides are very similar in properties such as boiling points and solubility, separation of N-vinylcarboxamides from the mixture is very difficult.

To address this problem, many methods have been developed for the purification of N-vinylcarboxamides. Patent Document 1 discloses extraction separation using water and aromatic hydrocarbons to solve difficult separation of unreacted materials by distillation. Patent Document 2 discloses separation by cooling crystallization with an organic solvent mixture. Patent Document 3 discloses extraction with an aqueous inorganic salt solution and an aromatic hydrocarbon.

However, these processes have been unable to produce N-vinylcarboxamides with sufficiently high purity.

Patent Documents 4 and 5 disclose purification by pressure crystallization capable of producing highly polymerizable N-vinylacetamides with relatively high purity.

However, the pressure crystallization entails a massive capital investment in facilities, and inexpensive supply of industrial products is difficult unless on a large scale.

Patent Document 1: JP-A-S61-289069

Patent Document 2: JP-A-S63-132868

Patent Document 3: JP-A-H02-188560

Patent Document 4: JP-A-H07-089916

Patent Document 5: JP-A-H07-089917

### DISCLOSURE OF THE INVENTION

It is an object of the invention to provide a low cost process for producing high-purity N-vinylcarboxamides having high polymerizability.

The present inventors studied diligently to solve the aforesaid problems. Consequently, it has been found that treatment with a specific alcohol and a specific aliphatic hydrocarbon produces high-purity N-vinylcarboxamides. The invention has been completed based on the finding.

The present invention is summarized as follows.

[1] A process for producing a high-purity N-vinylcarboxamide comprising:

(A) a step of dissolving a crude N-vinylcarboxamide in an alcohol having 1 to 3 carbon atoms, the crude N-vinylcarboxamide containing 50 to 97% by mass of an N-vinylcarboxamide;

(B) a step of adding an aliphatic hydrocarbon having 5 to 10 carbon atoms to the composition obtained in the step (A) to precipitate a crystal of the N-vinylcarboxamide; and

(C) a step of separating the crystal of the N-vinylcarboxamide precipitated in the step (B).

[2] The process for producing the high-purity N-vinylcarboxamide as described in [1], wherein in the step (A), the crude N-vinylcarboxamide is dissolved in an alcohol having 1 to 3 carbon atoms at 30 to 100° C.

[3] The process for producing the high-purity N-vinylcarboxamide as described in [1], wherein in the step (B), the crystal of the N-vinylcarboxamide is precipitated at −30 to 40° C.

[4] The process for producing the high-purity N-vinylcarboxamide as described in [1], wherein in the step (C), the crystal of the N-vinylcarboxamide is separated by filtration.

[5] The process for producing the high-purity N-vinylcarboxamide as described in [1] or [2], wherein the alcohol is methanol.

[6] The process for producing the high-purity N-vinylcarboxamide as described in [1] or [3], wherein the aliphatic hydrocarbon is n-hexane and/or petroleum ether.

[7] A high-purity N-vinylcarboxamide produced by the process described in any one of [1] to [6].

[8] An N-vinylcarboxamide (co)polymer produced by polymerizing a monomer that includes the high-purity N-vinylcarboxamide described in [7].

[9] The process for producing the high-purity N-vinylcarboxamide as described in any one of [1] to [6], wherein the N-vinylcarboxamide is N-vinylacetamide.

[10] The high-purity N-vinylacetamide produced by the process described in [9].

[11] An N-vinylacetamide (co)polymer produced by polymerizing a monomer that includes the high-purity N-vinylacetamide described in [10].

### EFFECTS OF THE INVENTION

The process according to the present invention enables inexpensive production of high-purity N-vinylcarboxamides having excellent polymerizability.

### BEST MODE FOR CARRYING OUT THE INVENTION

The present invention will be described in detail hereinbelow.

### Process for Producing High-Purity N-Vinylcarboxamides

The process for producing a high-purity N-vinylcarboxamide according to the present invention treats a crude N-vinylcarboxamide with a specific alcohol and a specific aliphatic hydrocarbon, and consequently the high-purity N-vinylcarboxamide is obtained with a purity higher than 97% by mass.

In the step (A), the crude N-vinylcarboxamide containing 50 to 97% by mass of an N-vinylcarboxamide is dissolved in an alcohol having 1 to 3 carbon atoms. When the crude N-vinylcarboxamide includes alcohol-insoluble components in this dissolving step, the alcohol-insoluble components may be removed by filtration preliminary.

Examples of the N-vinylcarboxamides include N-vinylacetamide, N-vinyl-N-methylacetamide, N-vinylformamide and N-methyl-N-vinylformamide. Of these, N-vinylacetamide is preferable, and the process of the invention can provide the high-purity N-vinylacetamide with a purity higher than 97% by mass.

Herein, the crude N-vinylcarboxamide refers to N-vinylcarboxamides containing impurities.

The crude N-vinylcarboxamides are producible by known methods. For example, they may be obtained by pyrolysis or catalytic cracking of N-(1-alkoxyethyl)carboxamides synthesized from carboxamides, acetaldehydes and alcohols (see JP-A-S50-7 6015). Alternatively, they may be obtained by pyrolysis of ethylidenebiscarboxamides synthesized from carboxamides and acetaldehydes (see JP-A-S61-106546).

Examples of the impurities include, although not particularly limited to, unreacted materials mixed during the production of the crude N-vinylcarboxamides. Specific examples include alcohols having 5 or less carbon atoms, carboxamides, N-(1-alkoxyethyl)carboxamides and ethylidenebiscarboxamides.

The crude N-vinylcarboxamide desirably contains 50 to 97% by mass, preferably 70 to 97% by mass of the N-vinylcarboxamide. If the N-vinylcarboxamide accounts for less than 50% by mass, the yield of the N-vinylcarboxamide is often low, and the N-vinylcarboxamide obtained tends to have low purity and unsatisfactory polymerizability.

When the crude N-vinylcarboxamide contains 50 to 70% by mass of the N-vinylcarboxamide, it may be subjected to the process of the invention as it is. Alternatively, the crude may be subjected to distillation or extraction to increase the concentration of the N-vinylcarboxamide. The alcohol-insoluble components in the crude N-vinylcarboxamide may be removed by such purification. Such purification leads to an improved yield of the N-vinylcarboxamide by the process of the invention, and is desirable from the viewpoints of purity and polymerizability.

The alcohols having 1 to 3 carbon atoms are used in the step (A), as discussed previously. When alcohols having 4 or more carbon atoms are used, the N-vinylcarboxamide tends not to be dissolved and purification often fails.

Examples of the alcohols having 1 to 3 carbon atoms include methanol, ethanol, n-propyl alcohol and isopropyl alcohol. The alcohols may be used singly or in combination of two or more kinds. Of the above alcohols, methanol is preferable. If the crude N-vinylcarboxamide is synthesized via an N-(1-alkoxyethyl)carboxamide, the same alcohol as the by-product alcohol is preferably used to simplify the process.

In view of efficiency, the alcohols are preferably used in minimum amounts that allow for dissolution of the crude N-vinylcarboxamide. The amount of the alcohols is preferably 0.01 to 2 parts by mass, more preferably 0.1 to 1 part by mass based on 1 part by mass of the crude N-vinylcarboxamide inclusive of the impurities. This amount of the alcohols is inclusive of alcohols mixed in a previous step if any. When the amount is less than described, the crystallization results in solidification of the entire system inclusive of the impurities and consequently the separation is often impossible. When the amount is far above that described, the precipitation of the N-vinylcarboxamide often fails.

The crude N-vinylcarboxamide is preferably dissolved in the alcohols at 30 to 100° C., more preferably 40 to 100° C. The temperatures in this range increase the solubility and allow for reduction of the alcohols used. Temperatures higher than described above can denature the N-vinylcarboxamides.

Water may be used as required together with the alcohols in order to facilitate spontaneous separation of the system into the alcohol phase and the aliphatic hydrocarbon phase in the step (B). The amount of water is preferably in the range of 0.1 to 50% by mass, more preferably 0.1 to 30% by mass of the total of the crude N-vinylcarboxamide, alcohols and water. When the amount of water exceeds this range, the N-vinylcarboxamide is not often precipitated and tends to be hydrolyzed.

In the production process of the invention, raw material tanks, filtrate tanks and product containers are desirably purged with an atmosphere of nitrogen or dry air. Small amounts of desiccants such as magnesium sulfate may be added to the crude N-vinylcarboxamide, whereby the N-vinylcarboxamide is prevented from absorbing moisture in the air and being gradually hydrolyzed.

In the production process of the invention, it is preferable that basic compounds be previously added to the crude N-vinylcarboxamide, whereby the tendency of the N-vinylcarboxamide to be hydrolyzed by acids in the presence of water is inhibited.

Examples of the basic compounds include sodium salts such as sodium carbonate, sodium hydrogen carbonate, sodium hydroxide, sodium (hydrogen) phosphate and sodium acetate; potassium salts such as potassium carbonate, potassium hydrogen carbonate, potassium hydroxide, potassium (hydrogen) phosphate and potassium acetate; and aromatic amines such as N-phenyl-α-naphthylamine, 4,4′-bis(α,α-dimethylbenzyl)diphenylamine, N-phenyl-N′-(1,3-dimethylbutyl)-p-phenylenediamine, N-phenyl-N′-isopropyl-p-phenylenediamine, N-phenyl-N′-(1-methylheptyl)-p-phenylenediamine, N-phenyl-N′-cyclohexyl-p-phenylenediamine, N,N′-diphenyl-p-phenylenediamine, N,N′-di-β-naphthyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine and N-phenyl-N′-(p-toluenesulfonyl)-p-phenylenediamine. Of these, the sodium salts are preferable, and sodium hydrogen carbonate is more preferable.

The basic compounds are desirably used in amounts of 1 to 10,000 ppm, preferably 10 to 1,000 ppm with respect to the crude N-vinylcarboxamide. Amounts of the inorganic salts exceeding this range tend to result in incomplete dissolution and will not provide corresponding effects. In the case of the aromatic amines, amounts thereof exceeding the above range tend to result in that the aromatic amine is not completely removed in the production steps and the N-vinylcarboxamide obtained has lower polymerizability. Amounts of the basic compounds less than described tend to fail to provide effects as stabilizers.

In the step (B), an aliphatic hydrocarbon having 5 to 10 carbon atoms is added to the composition obtained in the step (A) to precipitate a crystal of the N-vinylcarboxamide. Here, the composition comprises the N-vinylcarboxamide and the impurities dissolved in the alcohol, and the alcohol.

Preferred examples of the aliphatic hydrocarbons having 5 to 10 carbon atoms include alkanes having 5 to 10 carbon atoms, and cycloalkanes having 5 to 10 carbon atoms. Alkanes having 4 or less carbon atoms and cycloalkanes having 4 or less carbon atoms are often inconvenience because they are gas state at ambient temperatures. Alkanes having 11 or more carbon atoms and cycloalkanes having 11 or more carbon atoms with high boiling points tend not to be removed after the purification.

Examples of the alkanes having 5 to 10 carbon atoms include n-pentane, isopentane, neopentane; n-hexane, 2-methylpentane, 3-methylpentane; 2,3-dimethylbutane, 2,2-dimethylbutane; n-heptane and isomers thereof; n-octane and isomers thereof; n-nonane and isomers thereof; and n-decane and isomers thereof.

Examples of the cycloalkanes having 5 to 10 carbon atoms include cyclopentane; cyclohexane, methylcyclopentane; cycloheptane and isomers thereof; cyclooctane and isomers thereof; cyclononane and isomers thereof; and cyclodecane and isomers thereof.

The aliphatic hydrocarbons may be used singly or in combination of two or more kinds.

Of the above aliphatic hydrocarbons, n-hexane, isomers thereof and cyclohexane are preferable, and n-hexane is more preferable in view of operating temperature and viscosity of the aliphatic hydrocarbon phase when it is separated. Compositions such as petroleum ethers are preferable, and mixtures of n-hexane and petroleum ethers are also preferable.

The amount of the aliphatic hydrocarbons is preferably in the range of 1 to 50 parts by mass, more preferably 2 to 30 parts by mass based on 1 part by mass of the alcohols in the composition obtained in the step (A). Amounts of the aliphatic hydrocarbons less than described above tend to cause inefficient precipitation of the N-vinylcarboxamide. Amounts thereof exceeding the above range tend not to increase the precipitation efficiency.

The crystal of the N-vinylcarboxamide is preferably precipitated at −30 to 40° C., more preferably −25 to 30° C., more preferably −20 to 10° C. The N-vinylcarboxamide is not often precipitated at temperatures higher than described above.

When the crystal of the N-vinylcarboxamide is precipitated, the alcohol and the aliphatic hydrocarbon preferably form a two-phase system. When they form a two-phase system, the N-vinylcarboxamide that is dissolved in the alcohol phase is precipitated at the aliphatic hydrocarbon phase as a scale crystal. When they form a two-phase system, cooling the two-phase solution increases the purity of the N-vinylcarboxamide obtained.

In the step (C), the crystal of the N-vinylcarboxamide precipitated in the step (B) is separated.

The crystal of the N-vinylcarboxamide may be separated by evaporating the alcohol and the aliphatic hydrocarbon. Filtration is preferable to achieve efficient separation.

When the alcohol and the aliphatic hydrocarbon form a two-phase system, the crystal may be isolated by evaporating the solvents or by filtration without separating the alcohol phase and the aliphatic hydrocarbon phase. Preferably, the alcohol phase and the aliphatic hydrocarbon phase are separated and the crystal is separated from the aliphatic hydrocarbon phase. Although evaporating the aliphatic hydrocarbon gives the crystal, filtration is preferable because it removes impurities as well.

The process of the invention may be repeated to increase the purity of the N-vinylcarboxamide. Substances inhibiting the polymerization may be removed by hydrogenation or the like.

The alcohol phase includes materials used in the synthesis of the N-vinylcarboxamide, such as alcohols, carboxamides, N-(1-alkoxyethyl)carboxamides and ethylidenebiscarboxamides. These materials may be recovered and reused in the steps of producing the crude N-vinylcarboxamide, for example in the synthesis of N-(1-alkoxyethyl)carboxamide, the synthesis of ethylidenebiscarboxamide, and the synthesis of N-vinylcarboxamide from N-(1-alkoxyethyl)carboxamide or ethylidenebiscarboxamide. The residual N-vinylcarboxamide in the alcohol phase may be recovered by pressure crystallization, cooling crystallization or distillation.

### N-Vinylcarboxamide (Co)Polymer

The N-vinylcarboxamide (co)polymer according to the present invention may be produced by polymerizing a monomer that includes the high-purity N-vinylcarboxamide obtained by the above-described process. The (co)polymer is preferably produced by polymerizing a monomer that includes high-purity N-vinylacetamide obtained by the above-described process, that is, the (co)polymer is preferably an N-vinylacetamide (co)polymer. As used herein, the term N-vinylcarboxamide (co)polymer means a homopolymer obtained by polymerizing the N-vinylcarboxamide monomer, or a copolymer obtained by polymerizing the N-vinylcarboxamide monomer and another monomer. The (co)polymer is dissoluble in water and has a variety of uses.

Examples of the comonomers include (meth)acrylic acid monomers such as (meth)acrylic acid, salts thereof, methyl (meth)acrylate, ethyl (meth)acrylate, (iso)propyl (meth)acrylate, butyl (meth)acrylate, octyl (meth)acrylate, 2-ethylhexyl (meth)acrylate, methoxyethyl (meth)acrylate, 2-hydroxyethyl (meth)acrylate, 2-hydroxypropyl (meth)acrylate and polyoxyalkylene glycol mono(meth)acrylates; (meth)acrylamide monomers such as (meth)acrylamide, N,N-dimethyl(meth)acrylamide, N,N-diethyl(meth)acrylamide, N-methylol(meth)acrylamide, 2-(meth)acrylamide-2-methylpropanesulfonic acid and salts thereof, and N-isopropyl(meth)acrylamide; vinyl ester monomers such as vinyl acetate, vinyl butyrate and vinyl valerate; styrene monomers such as styrene, α-methylstyrene, p-methylstyrene, p-methoxystyrene and m-chlorostyrene; vinyl ether monomers such as methyl vinyl ether, butyl vinyl ether and vinyl benzyl ether; dicarboxylic acid monomers such as maleic anhydride, maleic acid and salts thereof, fumaric acid and salts thereof, dimethyl maleate and diethyl fumarate; allyl monomers such as allyl alcohols, allyl phenyl ethers and allyl acetates; (meth)acrylonitriles, vinyl chloride, ethylene and propylene.

The comonomers may be used singly or in combination of two or more kinds. The amount of the comonomers may be determined appropriately depending on the use of the copolymer, and is desirably not more than 60% by mass, preferably not more than 40% by mass with respect to the total amount of the monomers.

### Uses of N-Vinylcarboxamide (Co)Polymer

The (co)polymer has thickening and dispersing effects and is suitably used in a wide range of fields as described below.

(1) Industrial Dispersants

The (co)polymer maybe used as dispersants for inorganic and organic powders. Examples of the powders include inorganic powders such as silica, alumina, titania and calcium carbonate; mineral powders such as talc and kaolin; pigment powders such as carbon blacks; resin powders such as polyurethane, polyacrylate and polyethylene; and organic powders such as stearates. The (co)polymer disperses these powders in polar solvents such as water.

(2) Thickeners and Dispersants for Paints and Inks

The (co)polymer may be used as dispersants, viscosity modifiers, leveling agents and wetting agents for paints and inks.

(3) Treatment Agents and Extracting Agents for Water and Oil

(4) Cosmetics

The (co)polymer may be used as emulsion stabilizers, lubricants and emulsifying agents (for emulsion cosmetics) in cosmetics such as shampoos, conditioners and lotions. The (co)polymer may also be used in mask packs and styling agents.

(5) Toiletries

The (co)polymer may be used as thickeners for liquid detergents (for clothing materials, kitchens, bathrooms and tiles), toothpastes, cleansers, softeners and industrial detergents.

(6) Adhesives and Adhesive Auxiliaries

(7) Medicals

The (co)polymer may be used for the purposes of holding medical agents and sustaining the release of medical agents. Specifically, the (co)polymer finds use in tablets (sustained-release tablets), enteric-coated preparations, bases of adhesive preparations such as cataplasms and plasters, ointments, controlled-release preparations, intragastric buoyant sustained-release tablets, mucosal preparations, dermatological compositions (medical films), wound coverings, dental materials, oral absorbents and interdental cleaning materials. The (co)polymer may also be used as lubricants for medical instruments such as urethral catheters and enemas that are heated in sterilizing autoclaves for reuse, and may be used as viscosity modifiers for diagnostic agents.

(8) Water Absorption Materials, Water Retention Agents, Sealing Agents and Cold Insulators

(9) Others

The (co)polymer may be used as treatment agents in papermaking, air fresheners, deodorants, desiccants, fermentation assistants, releasing agents for packing materials and old wallpaper, and thickeners for toys, sweat cloth materials, ultrasonic crack inspection contact media, ultrasonic probes and electrolyte substrates of batteries and sensors.

The present invention will be described in greater detail by examples below, but it should be construed that the invention is in no way limited thereto.

### EXAMPLES

### Example 1

N-(1-methoxyethyl) acetamide was pyrolyzed at 350° C., and low-boiling fractions were evaporated to obtain a crude N-vinylacetamide (which contained 86.6% by mass of N-vinylacetamide, 3.5% by mass of acetamide and 9.4% by mass of N-(1-methoxyethyl)acetamide). 50 g of the crude N-vinylacetamide was combined with 10 g of methanol, and the mixture was heated to 50° C. to dissolve the crude N-vinylacetamide. The resulting solution was combined with 200 g of n-hexane, followed by cooling to 10° C. to precipitate a crystal. The crystal of the N-vinylacetamide was white in color and scale in shape, and was precipitated at the upper n-hexane phase. The lower methanol phase was withdrawn, and the crystal was recovered by filtration. The crystal weighed 20.6 g and was 98.7% in purity.

### Example 2

N-(1-methoxyethyl) acetamide was pyrolyzed at 330° C., and low-boiling fractions were evaporated to obtain a crude N-vinylacetamide (which contained 85.2% by mass of N-vinylacetamide, 3.3% by mass of acetamide and 11.2% by mass of N-(1-methoxyethyl) acetamide). The crude N-vinylacetamide was treated as described in Example 1 to produce a crystal of the N-vinylacetamide, which weighed 16.3 g and was 99.1% in purity.

### Example 3

The procedures of Example 1 were repeated except that n-hexane was replaced by petroleum ether. Consequently, a crystal of N-vinylacetamide was precipitated, which was white in color and scale in shape and was precipitated at the upper petroleum ether phase. The N-vinylacetamide crystal was filtered without separating the methanol phase and the petroleum ether phase. The crystal was cleaned with a very small amount of cold methanol. The crystal obtained weighed 13.9 g and was 98.3% in purity.

### Comparative Example 1

Purification of N-vinylacetamide was attempted in the same manner as in Example 1 except that n-hexane was not added. Crystallization did not take place when the solution of the crude N-vinylacetamide was cooled at 10° C. Crystallization took place at not more than −5° C. but resulted in solidification of the entire system to disenable recovery of the N-vinylacetamide crystal by filtration or whatsoever.

### Comparative Example 2

Purification of N-vinylacetamide was attempted in the same manner as in Example 1 except that 200 g of n-hexane replaced 10 g of methanol for dissolving the crude N-vinylacetamide. The crude N-vinylacetamide was not dissolved even by heating at 50° C., and the phase of the melted crude N-vinylacetamide containing impurities and the phase of n-hexane existed separately. Consequently, crystallization of N-vinylacetamide failed.

### Example 4

240 g of the high-purity N-vinylacetamide obtained in Example 1 was dissolved in 160 g of pure water, followed by addition of 1.2 g of thioglycolic acid as chain transfer agent. The solution was neutralized at a pH of 9 to 10 with an aqueous sodium hydroxide solution. Consequently, a monomer solution was prepared. Separately, an initiator solution was prepared by dissolving 4.8 g of azobis radical polymerization initiator 2,2′-azobis(2-methylpropionamidine) dihydrochloride (V-50 manufactured by Wako Pure Chemical Industries, Ltd., molecular weight: 271, capable of generating 2 molar equivalents of radical species upon cleavage) in 395.2 g of pure water. The monomer solution was stored at not more than 10° C. to prevent polymerization. A 1-liter flask equipped with a condenser tube, a thermometer, a stirrer and a dropping device was charged with 200 g of water, and the water was heated under reflux at approximately 100° C. in a stream of nitrogen. The monomer solution and the initiator solution were added dropwise simultaneously over a period of about 1 hour, and the N-vinylacetamide was polymerized. The weight-average molecular weight of the poly (N-vinylacetamide) was determined to be 45,000 by the light-scattering method. Polymerizability of the N-vinylacetamides was as high as that of N-vinylacetamides obtained by pressure crystallization as disclosed in Patent Documents 4 and 5.

When the specific alcohols and the specific aliphatic hydrocarbons as described above are not used, the purified product is not obtained, or the yield of the purified product is low and impractical.

1. A process for producing a high-purity N-vinylcarboxamide comprising:
(A) a step of dissolving a crude N-vinylcarboxamide in an alcohol having 1 to 3 carbon atoms, the crude N-vinylcarboxamide containing 50 to 97% by mass of an N-vinylcarboxamide;
(B) a step of adding an aliphatic hydrocarbon having 5 to 10 carbon atoms to the composition obtained in the step (A) to precipitate a crystal of the N-vinylcarboxamide; and
(C) a step of separating the crystal of the N-vinylcarboxamide precipitated in the step (B).

2. The process for producing the high-purity N-vinylcarboxamide as claimed in claim 1, wherein in the step (A), the crude N-vinylcarboxamide is dissolved in an alcohol having 1 to 3 carbon atoms at 30 to 100° C.

3. The process for producing the high-purity N-vinylcarboxamide as claimed in claim 1, wherein in the step (B), the crystal of the N-vinylcarboxamide is precipitated at −30 to 40° C.

4. The process for producing the high-purity N-vinylcarboxamide as claimed in claim 1, wherein in the step (C), the crystal of the N-vinylcarboxamide is separated by filtration.

5. The process for producing the high-purity N-vinylcarboxamide as claimed in claim 1, wherein the alcohol is methanol.

6. The process for producing the high-purity N-vinylcarboxamide as claimed in claim 1 wherein the aliphatic hydrocarbon is n-hexane and/or petroleum ether.

7. A high-purity N-vinylcarboxamide produced by the process according to claim 1.

8. An N-vinylcarboxamide (co)polymer produced by polymerizing a monomer that includes the high-purity N-vinylcarboxamide according to claim 7.

9. The process for producing the high-purity N-vinylcarboxamide as claimed in claim 1, wherein the N-vinylcarboxamide is N-vinylacetamide.

10. A high-purity N-vinylacetamide produced by the process according to claim 9.

11. An N-vinylacetamide (co)polymer produced by polymerizing a monomer that includes the high-purity N-vinylacetamide according to claim 10.

12. The process for producing the high-purity N-vinylcarboxamide as claimed in claim 2, wherein the alcohol is methanol.

13. The process for producing the high-purity N-vinylcarboxamide as claimed in claim 3, wherein the aliphatic hydrocarbon is n-hexane and/or petroleum ether.

14. A high-purity N-vinylcarboxamide produced by the process according to claim 2.

15. A high-purity N-vinylcarboxamide produced by the process according to claim 3.

16. A high-purity N-vinylcarboxamide produced by the process according to claim 4.

17. A high-purity N-vinylcarboxamide produced by the process according to claim 5.

18. A high-purity N-vinylcarboxamide produced by the process according to claim 6.

19. A high-purity N-vinylcarboxamide produced by the process according to claim 12.

20. A high-purity N-vinylcarboxamide produced by the process according to claim 13.
